(19) United States
(12) Patent Application Publication
(10) Pub. No.: US 2020/0077781 A1
(43) Pub. Date: Mar. 12, 2020

(54) TONGUE BRUSH HAVING BRISTLES EMANATING FROM ONE SURFACE AND A TONGUE SCRAPER EMANATING FROM AN OPPOSING SURFACE

(71) Applicant: Dr Bobs Inc, Springville, UT (US)

(72) Inventor: Robert K. Wagstaff, Springville, UT (US)

(21) Appl. No.: 16/123,805

(22) Filed: Sep. 6, 2018

(51) Int. Cl. A46B 15/00; A46B 9/06

(52) U.S. Cl. CPC A46B 15/0081; A61B 17/244; A46B 9/06

(57) ABSTRACT

A tongue brush includes bristles that emanate from a front surface of the head of the tongue brush and a tongue scraper that emanates from the opposing rear surface of the head of the tongue brush. These bristles can include tongue brush bristles as well as tufted bristles. The tongue brush bristles may be arranged in a first region of the front surface separately from the tufted bristles. The tongue scraper can extend in an opposite direction and from an opposing surface from the bristles.

### CROSS-REFERENCE TO RELATED APPLICATIONS

Not Applicable.

### BACKGROUND

Toothbrushes typically include tufted bristles because the tufted bristles are effective for cleaning the substantially smooth surface of teeth. Tufted bristles, however, are not effective for cleaning the uneven surface of the tongue since the tufts are incapable of reaching into the crevices where much of the bacteria and food particles are located. For this reason, various tongue cleaning devices have been created. These tongue cleaning devices may include tongue brush bristles and/or a tongue scraper. As opposed to tufted bristles, which are commonly formed of nylon, tongue brush bristles are typically formed of plastic (e.g., polyethylene) or rubber to allow the tongue brush bristles to be formed in various shapes and sizes. The tongue brush bristles are typically pointed and spaced so that the individual bristles can reach into the crevices of the tongue. The flexible pointed bristles of tongue brush bristles are effective in loosening the bacteria and food particles that are present on the soft surface of the tongue. It has been reported that up to 90% of bad breath comes from the tongue so the best way to eliminate bad breath is to remove the cause of bad breath rather than try to cover it up with gum, mints, or mouthwash.

### BRIEF SUMMARY

The present invention extends to a tongue brush that includes bristles that emanate from a front surface of the head of the tongue brush and a tongue scraper that emanates from the opposing rear surface of the head of the tongue brush. These bristles can include tongue brush bristles as well as tufted bristles. The tongue brush bristles may be arranged in a first region of the front surface separately from the tufted bristles. The tongue scraper extends in an opposite direction and from an opposing surface from the bristles.

In some embodiments, the present invention is implemented as a tongue brush that includes: a head having a proximal end, a distal end, a front surface and a rear surface opposite the front surface; tongue brush bristles that emanate from the front surface; tufted bristles that also emanate from the front surface; and a tongue scraper that emanates from the rear surface.

In other embodiments, the present invention is implemented as a tongue brush that includes: a head having a proximal end, a distal end, a front surface and a rear surface opposite the front surface; tongue brush bristles that emanate from the front surface, the tongue brush bristles being arranged in a proximal region on the front surface; tufted bristles that also emanate from the front surface, the tufted bristles being positioned distal to the proximal region; and a tongue scraper that emanates from the rear surface.

In other embodiments, the present invention is implemented as a tongue brush that includes: a head having a proximal end, a distal end, a front surface and a rear surface opposite the front surface; tongue brush bristles that emanate from the front surface; tufted bristles that also emanate from the front surface, the tufted bristles being arranged in a row that spans a width of the front surface; and a tongue scraper that emanates from the rear surface, the tongue scraper forming a portion of the distal end of the head.

This summary is provided to introduce a selection of concepts in a simplified form that are further described below in the Detailed Description. This Summary is not intended to identify key features or essential features of the claimed subject matter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates front perspective view of a brush that is configured in accordance with embodiments of the present invention;

FIG. 2 illustrates a side view of the brush of FIG. 1;

FIG. 3 illustrates a rear perspective view of the brush of FIG. 1;

FIG. 4 illustrates a distal end view of the brush of FIG. 1; and

FIG. 4A illustrates a distal end view of a brush that includes a tongue scraper having teeth.

### DETAILED DESCRIPTION

In this specification and claims, the term tongue brush should be construed to encompass any type of brush whether or not the brush includes a handle. The head of the brush will be described as being at the distal end of the brush, whereas the handle (if the brush includes one) will be described as being at the proximal end of the brush. The front surface of the head will refer to the surface of the head from which the bristles emanate, while the rear surface of the head will refer to the opposing surface from which the tongue scraper emanates.

A tongue brush bristle should be construed as a non-tufted bristle formed of a plastic material (e.g., polyethylene) that has a base that is coupled to the head and a tip opposite the base. The base of a tongue brush bristle is wider than the tip with the tip forming a point. In other words, the width/diameter of a tongue brush bristle tapers from the base to the tip. This tapering gives a tongue brush bristle rigidity at the base and flexibility at the tip so that the pointed tip can insert into crevices in the tongue to remove food particles and bacteria. The flexible pointed tip may also enable the tongue brush bristles to insert between teeth and along the gum line when the brush is used to brush the teeth thereby enhancing the brush's effectiveness for removing bacteria, food particles, and/or soft plaque from the teeth.

Tufted bristles are thin strands that are typically formed of nylon and clustered together into tufts. The tufted bristles employed on a brush of the present invention can be similar to any of the tufted bristles commonly used on toothbrushes. In the figures, the tufted bristles are all shown as having substantially the same length. However, in embodiments of the present invention, the tufted bristles can have varying heights as is known in the art.

The handle and head of the brush can typically be formed of a plastic material (e.g., polypropylene) that is different from the plastic material from which the tongue brush bristles are formed (e.g., polyethylene). The head of a brush configured in accordance with the present invention can have any suitable shape and/or size. The tufted bristles can be coupled to the head in any manner known in the art. In some embodiments, the tongue brush bristles can be formed as part of a common base that is embedded within or coupled to the head. For example, the tongue brush bristles and their base can be molded as a single component into the head, or can be molded separately from the head and then coupled to the head using any suitable coupling technique.

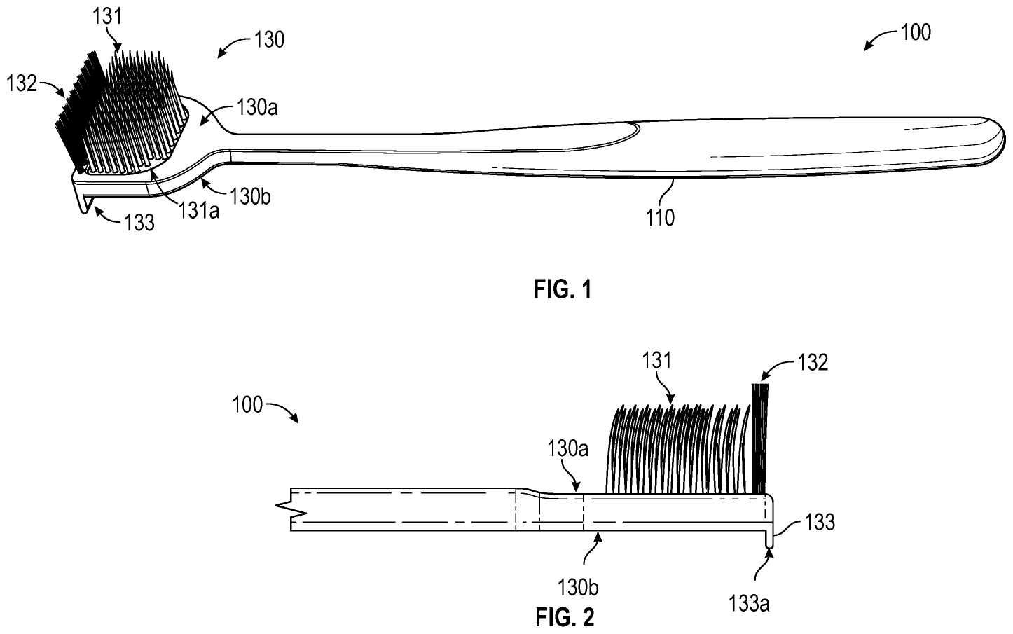

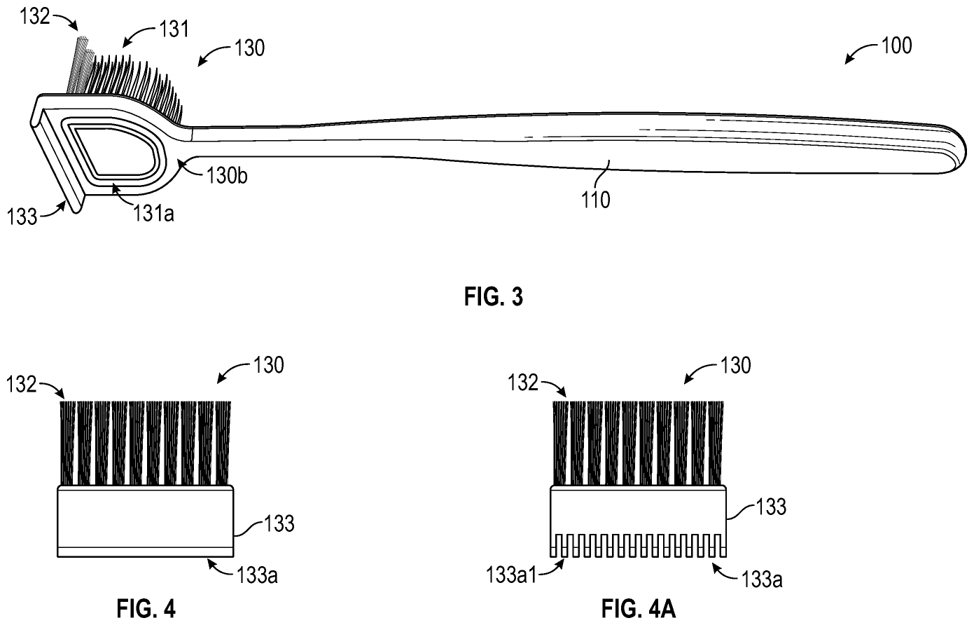

FIGS. 1-4 illustrate various view of a brush 100 that is configured in accordance with one or more embodiments of the present invention. Brush 100 comprises a handle 110 and a head 130 coupled to the distal end of handle 110. Although brush 100 is in the general form of a toothbrush, a brush could be configured in other manners. For example, handle 110 could be removed or replaced with a different shaped handle. Head 130 can preferably be shaped and sized to allow it to be placed within the human mouth to scrub and scrape the tongue.

Brush 100 includes both tongue brush bristles 131 and tufted bristles 132 that emanate from front surface 130a of head 130. Tongue brush bristles 131 extend from a base 131a that is integrated into head 130 and that may extend through to rear surface 130b as shown in FIG. 4. Each tongue brush bristle 131 is tapered from its base to its tip such that the tip forms a point. In some embodiments, the tip of each tongue brush bristle 131 may be curled as is shown in the figures. However, the tips may also be straight. Also, although the figures depict the tips as generally curling in the same direction, the tips may be curled in different directions.

Tongue brush bristles 131 are arranged within a proximal region of front surface 130a which spans substantially across a width of front surface 130a. Additionally, a row of tufted bristles 132 is positioned distal to tongue brush bristles 131. This row also spans substantially across the width of front surface 130a. As is best seen in FIG. 2, the row of tufted bristles 132 is positioned at the distal end of head 130.

This arrangement of tongue brush bristles 131 in a region proximal to a row of tufted bristles 132 provides enhanced cleaning potential for the surface of the tongue. In particular, as head 130 is moved back and forth within the mouth, tongue brush bristles 131 can insert into crevices in the surface of the tongue and loosen material while tufted bristles 132 can pass over and enhance the removal of the material from the crevices and surface of the tongue. As depicted, the length of tufted bristles 132 may exceed the length of tongue brush bristles 131 to ensure that both types of bristles can remain in contact with the surface of the tongue during use.

Brush 100 also includes a tongue scraper 133 that emanates from rear surface 130b in a direction opposite that of tongue brush bristles 131 and tufted bristles 132. Tongue scraper 133 can be formed of the same material as head 130 and handle 110 (e.g., polypropylene). For example, as shown in the figures, tongue scraper 133 can be molded as a protruding portion of rear surface 130b. In some embodiments, the height and width of tongue scraper 133 can be 3 mm and 1 mm respectively. The edge 133a of tongue scraper 133 may be given a slightly rounded shape so that edge 133a will not cut into the tongue.

Although tongue scraper 133 is shown as forming a portion of the distal end of head 130, in other embodiments, tongue scraper 133 may be positioned proximally away from the distal end of head 130. Also, although tongue scraper 133 is shown as spanning the entire width of rear surface 130b, in some embodiments, tongue scraper 133 may span less than or more than the entire width of rear surface 130b.

Because tongue scraper 133 emanates from rear surface 130b opposite from front surface 130a, tongue scraper 133 can be used to scrape the surface of the tongue separately from, and oftentimes after, cleaning the tongue with tongue brush bristles 131 and tufted bristles 132. For example, a user may typically insert head 130 into his or her mouth with front surface 130a facing the tongue. In this orientation, the user can scrub the surface of the tongue to loosen and remove material from the crevices and the surface. Then, the user can rotate brush 130 so that rear surface 130b faces the tongue. In this orientation, the user can scrape the loosened material from the tongue by pulling tongue scraper 133 along the surface. This scraping will likely also remove and loosen additional material from the tongue's surface. This separate brushing and scraping can enhance the overall effectiveness of brush 100.

In FIGS. 1-4, the edge 133a of tongue scraper 133 is shown as being continuous. Such configurations will ensure that edge 133a contacts the largest possible area on the surface of the tongue. However, in some embodiments such as is shown in FIG. 4A, edge 133a may be notched to thereby form teeth 133a1 similar to the teeth of a comb. Teeth 133a1 can function to enable tongue scraper 133 to better reach into crevices or uneven surfaces in the tongue so that more material can be scraped from the tongue.

In addition to the arrangement of tongue brush bristles 131 and tufted bristles 132 shown in the figures, embodiments of brush 100 may include any other arrangement of tongue brush bristles 131 and tufted bristles 132. For example, U.S. Pat. No. 9,700,127, which is incorporated herein by reference, discloses various arrangements of tongue brush and tufted bristles any of which could be employed on embodiments of brush 100.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description.

1. A tongue brush comprising:
a head having a proximal end, a distal end, a front surface and a rear surface opposite the front surface, the front surface having a length and a width;
a region of tongue brush bristles that emanate from the front surface;
a row of tufted bristles that also emanate from the front surface, the tufted bristles being arranged in a row that spans the width of the front surface along the distal end of the head, wherein the region of tongue brush bristles is positioned proximal to but adjacent the row of tufted bristles, the region of tongue brush bristles spanning the width of the front surface and spanning the length of the front surface between the row of tufted bristles and the proximal end of the head; and
a tongue scraper that emanates from the rear surface.

2. The tongue brush of claim 1, wherein the tongue scraper and the head are formed of the same material.

3. The tongue brush of claim 2, wherein the tongue scraper and the head are formed of polypropylene.

4. The tongue brush of claim 1, wherein the tongue brush bristles are formed of a first plastic material and the tongue scraper and the head are formed of a second plastic material different from the first plastic material.

5. The tongue brush of claim 4, wherein the first plastic material is polyethylene and the second plastic material is polypropylene.

6. The tongue brush of claim 1, wherein the tongue scraper is formed at the distal end of the head.

7. The tongue brush of claim 1, wherein the tongue scraper is an extension of the distal end of the head.

8. The tongue brush of claim 1, wherein the tongue brush bristles are formed as part of a common base that is embedded within or coupled to the head.

9. The tongue brush of claim 8, wherein the common base extends through the head to the rear surface.

10. The tongue brush of claim 9, wherein the tufted bristles are longer than the tongue brush bristles.

11. The tongue brush of claim 1, wherein the tongue brush bristles each have a base that is coupled to the front surface and a tip opposite the base, the tip forming a point.

12. The tongue brush of claim 1, wherein the tufted bristles are longer than the tongue brush bristles.

13. The tongue brush of claim 1, further comprising:
a handle coupled to the head.

14. The tongue brush of claim 1, wherein an edge of the tongue scraper is continuous.

15. The tongue brush of claim 1, wherein an edge of the tongue scraper forms teeth.

16. A tongue brush comprising:
a head having a proximal end, a distal end, a front surface and a rear surface opposite the front surface, the front surface having a length and a width;
a handle coupled to and extending distally from the distal end of the head;
tongue brush bristles that emanate from the front surface, the tongue brush bristles being arranged in a proximal region on the front surface, the proximal region extending across the width of the front surface and across the length of the front surface;
tufted bristles that also emanate from the front surface, the tufted bristles being arranged in a row that is positioned distal to the proximal region and that extends along the distal end of the head across the width of the front surface, the tufted bristles being longer than the tongue brush bristles; and
a tongue scraper that emanates from the rear surface.

17. The tongue brush of claim 14, wherein the tongue scraper is an extension of the distal end of the head.

18. The tongue brush of claim 14, wherein an edge of the tongue scraper is notched to thereby form teeth.

19. A tongue brush comprising:
a head having a proximal end, a distal end, a front surface and a rear surface opposite the front surface, the front surface having a length and a width;
a region of tongue brush bristles that emanate from the front surface;
a row of tufted bristles that also emanate from the front surface, the tufted bristles being arranged in a row that spans the width of the front surface along the distal end of the head, wherein the region of tongue brush bristles is positioned proximal to but adjacent the row of tufted bristles, the region of tongue brush bristles spanning the width of the front surface and spanning the length of the front surface between the row of tufted bristles and the proximal end of the head; and
a tongue scraper that emanates from the rear surface, the tongue scraper forming an extension of the distal end of the head.

20. The tongue brush of claim 19, wherein the tongue scraper spans a width of the rear surface of the head.
